(19) United States
(12) Patent Application Publication
(10) Pub. No.: US 2018/0279936 A1
(43) Pub. Date: Oct. 4, 2018

(54) VIDEO DATABASES AND METHODS FOR DETECTION OR DIAGNOSIS OF NEURODEVELOPMENT DISORDERS

(71) Applicant: Tiranoff Productions LLC, Brooklyn, NY (US)

(72) Inventor: Louise TIRANOFF, Brooklyn, NY (US)

(21) Appl. No.: 15/562,872

(22) PCT Filed: Apr. 1, 2016
(86) PCT No.: PCT/US16/25581

§ 371 Date: Sep. 28, 2017

Related U.S. Application Data
(60) Provisional application No. 62/141,812, filed on Apr. 1, 2015.

(51) Int. Cl. A61B 5/16; A61B 5/00; A61B 5/11; G06F 17/30; G16H 50/20; G16H 50/70

(52) U.S. Cl. CPC A61B 5/167; A61B 5/168; A61B 5/4094; A61B 5/1124; G06K 9/00342; G06F 17/30858; G06F 17/3082; G16H 50/20; G16H 50/70; A61B 5/1128

(57) ABSTRACT

A database of video clips is provided wherein each video clip depicts normal or abnormal behavioral patterns in children aged from birth and older or in adults. The behavior patterns in the video clip database are relevant to neurodevelopmental disorders. The behavior patterns depicted in the video clips are indexed and searchable. Also provided is a method of assessing a behavioral abnormality in the test subject. The normal and abnormal patterns for a given behavior can be compared to a test subject performing the same behavior depicted in the video library, to permit a medical professional to assess the extent of a behavioral abnormality in the test subject.

### CROSS REFERENCE TO RELATED APPLICATIONS

This application claims priority to U.S. Patent Application No. 62/141,812, filed Apr. 1, 2015 and PCT International Application PCT/US2016/025581, filed Apr. 1, 2016, the entire contents of which are incorporated by reference.

### FIELD OF THE INVENTION

This invention pertains to a digital media platform application including a video-based natural histories database and other tools for the study of rare or difficult-to-diagnose neurodevelopmental disorders.

### BACKGROUND

Neurodevelopmental disorders are a group of disorders of the CNS which can manifest as neuropsychiatric problems, impaired language and non-verbal communication development and/or impaired motor function, which impacts on personal, social, academic or occupational functioning. Many of these disorders have a known genetic description, but for others, the genetic descriptions are unknown, or multiple genetic abnormalities are implicated.¹ Other neuropsychiatric disorders are adult onset, such as dementia syndromes like Alzheimer's Disease. ¹ See, e.g., Joseph D. Buxbaum, “Multiple rare variants in the etiology of autism spectrum disorders,” Dialogues Clin Neurosci. (2009) March; 11(1): 35-43.

Neurodevelopmental disorders are more fully described in the fifth revision of the American Psychiatric Association's Diagnostic and Statistical Manual of Mental Disorders 5th edition (DSM-5). Two commonly occurring neurodevelopmental disorders defined in the DSM-5 are autism spectrum disorder (ASD) and attention-deficit/hyperactivity disorder (ADHD). The spectrum of these disorders encompasses autism, Asperger syndrome, pervasive developmental disorder not otherwise specified (PDD-NOS), and childhood disintegrative disorder. One in 68 children in the United States has ASD. These disorders are characterized by social deficits and communication difficulties, stereotyped or repetitive behaviors and interests, sensory issues, and in some cases, cognitive delays. These characteristic signs and behaviors are difficult if not impossible to capture in written clinical notes.

Another significant neurodevelopment disorder is Angelman syndrome. This disorder is characterized by a stiff, jerky gait, characteristic arm movements, absent speech, excessive laughter, and seizures. Angelman Syndrome has a genetic cause. The Angelman syndrome gene (UBE3A) is located at chromosome 15, band q12. In the brain, the Angelman gene is primarily expressed from the maternally inherited chromosome 15. Abnormalities (or the absence) of this gene causes Angelman Syndrome. The incidence of Angelman Syndrome is estimated at between 1:10,000 and 1:20,000 births.

Another neurodevelopmental disorder is Fragile X syndrome. This disorder is caused by a defect in the FMR1 gene, located on the long arm of the X chromosome. At the start of this gene, a region of DNA varies in length from one person to another. When the stretch of DNA expands beyond a certain length, the gene is switched off and it does not function normally. Fragile X patients may exhibit intellectual disabilities, attention deficit disorder, unstable moods, speech delay, autistic behaviors, and seizures. The Fragile X mutation is estimated to occur in 1:4000 males and 1:6000 females. Genetic testing is available, and children who exhibit behavior disorders benefit from early intervention.

Another neurodevelopmental disorder is mucopolysaccharidosis I (MPS I). MPS I, along with six other MPS diseases is a mucopolysaccharide disease that is relentlessly progressive and potentially fatal. MPS I has also been called Hurler, Hurler-Scheie and Scheie syndrome. There is no cure for MPS diseases, but there are ways of managing and treating the problems they cause. Mucopolysaccharides are long chain sugar molecules used in the building of connective tissues in the body. There is a continuous process of replacing used materials and breaking them down for disposal. Children with MPS diseases are missing critical enzymes for this process. In MPS I, the enzyme alpha-L-iduronidase is missing. This enzyme is essential in breaking down the mucopolysaccharides called dermatan sulfate and heparan sulfate. The incompletely broken down mucopolysaccharides remain stored in cells in the body causing progressive damage. Babies may show little sign of the disease, but as more and more cells become damaged, symptoms start to appear. MPS I (Hurler-Scheie) is a continuum of severity based upon the symptoms, ranging from severe to attenuated. There is a great deal of variability of symptoms among individuals with MPS I, often making the specific designation difficult. Generally, severe MPS I will present within the first year of life while less severe (attenuated) forms present during childhood. Although individuals with attenuated MPS I have normal intelligence, they may have a variety of symptoms that can range from mild to severe.

Another neurodevelopmental disorder is mucopolysaccharidosis II (MPS II). This is a rare genetic disorder that affects many body systems and may lead to damage of different body organs. MPS II is also known as Hunter syndrome. It is caused by a defect in the gene that instructs the body to make the enzyme iduronate-2-sulfatase—also called I2S. Because of this defect, cells either produce the enzyme in low amounts or cannot produce it at all. The enzyme is needed to break down glycosaminoglycans (GAGs), If GAGs are not broken down, they build up in the cell, eventually leading to damage to cells, tissues, and organs. There are two types of Hunter syndrome: early onset (more severe) and late onset (a milder form). Hunter syndrome patients commonly (but not always) have intellectual disabilities and autistic-type symptoms that are amenable to early intervention.

Another neurodevelopmental disorder is Gaucher disease, a rare genetic disorder in which the enzyme called glucocerebrosidase is missing. This condition is also called Glucocerebrosidase deficiency or Glucosylceramidase deficiency. Gaucher disease affects an estimated 1 in 50,000 to 1 in 100,000 people in the general population. People of Eastern and Central European (Ashkenazi) Jewish heritage are more likely to have Gaucher disease. It is an autosomal recessive disease. This means that the mother and father must both pass an abnormal copy of the disease gene to their child in order for the child to develop the disease. A parent who carries an abnormal copy of the gene but doesn't have the disease is called a silent carrier. The lack of the glucocerebrosidase enzyme causes harmful substances to build up in the liver, spleen, bones, and bone marrow. These substances prevent cells and organs from working properly.

There are three main subtypes of Gaucher disease. Type 1 disease is most common. It involves bone disease, anemia, an enlarged spleen and low platelets (thrombocytopenia). Type I affects both children and adults. It is most common in the Ashkenazi Jewish population. Type 2 disease usually begins in infancy with severe neurologic involvement. This form can lead to rapid, early death. Type 3 disease may cause liver, spleen, and brain problems. People with this type may live into adulthood. Symptoms vary, but may include: Bone pain and fractures, cognitive impairment, easy bruising, enlarged spleen, enlarged liver, fatigue, heart valve problems, lung disease, seizures, severe swelling at birth, and skin changes. Some forms may include neurodevelopmental symptoms.

Early intervention and intensive therapy starting at an early age (less than 24 months) can arrest potentially debilitating life-long effects of many disorders with neurological impairments, including neurodevelopmental disorders such as ASD, and may be of significant benefit in Angelman Syndrome. These are not degenerative disorders, so patients may improve their living skills with support. Thus, early screening and early detection of children exhibiting Angelman or ASD symptoms is an important public health need.

Although ASD can be reliably detected in children less than 3 years, the average age of diagnosis is 4 to 5 years old, and later for children from minority groups. The American Academy of Pediatrics (AAP) has mandated autism-specific screening for all children using a validated screening tool at both the 18- and 24-month well-child exams, yet adoption of this mandate has been slow. Many pediatricians continue to report a lack of familiarity with ASD screening tools as well as a lack of time to conduct screening. Accordingly, only 8 to 28% of surveyed pediatricians report any use of an ASD screener, let alone routine or universal use.

ASD is detected in a young child when 1) parents observe something atypical in their child, and bring their concerns to their pediatric or family care provider 2) when a screening tool reveals that a child may be at risk for an ASD and 3) when a doctor observes the warning signs in a child during the well visit. Too often this process is haphazard and unreliable, and goes against AAP protocols for standardized screening and surveillance.

The Centers for Disease Control (CDC) advocate early screening for ASD with “Learn the Signs, Act Early.” Early intervention raises IQ and cognitive ability, reduces symptom severity, and leads to more favorable long-term functional outcomes, including successful educational inclusion. It also helps mitigate long-term financial costs. Lifetime costs from lost productivity of adults with ASD and parents place huge financial burdens on families and society.

Screening involves using standardized, validated tools to evaluate risk for the neurodevelopment disorders such as ASD. Most parents and many doctors lack familiarity with the diverse and subtle manifestations of these perplexing disorders. As such, screeners may be confusing to some parents and autism-specific surveillance a challenge to even the most dedicated primary care physician. Further, many parents and some doctors do not realize the importance of starting treatment as early as possible.

Some diagnostic characteristics of ASD include:
No babbling by 12 months.
No gesturing (pointing, waving, etc.) by 12 months.
No single words by 16 months.
No two-word (spontaneous, not just echolalic) phrases by 24 months.
Any loss of any language or social skills, at any age.

Screening tools for ASD include the “Modified Checklist for Autism in Toddlers” (called the “M-CHAT-R,” or “M-CHAT-R/F”), the Early Screening of Autistic Traits Questionnaire, and the First Year Inventory. The terms “MCHAT,” “M-CHAT-R,” and “M-CHAT-R/F” are used interchangeably in this disclosure, and all refer to variations of the diagnostic tool available on the website https://www.m-chat.org/.

The M-CHAT-R test consists of 20 yes/no questions designed to be administered to parents/guardians and interpreted by pediatric providers in the context of developmental surveillance. Two sample questions are “if you point at something across the room, does your child look at it?” and “Have you ever wondered if your child might be deaf?” Each child given the test receives a score indicating their risk of having a neurodevelopmental disorder. High risk results are assigned to a follow up evaluation and therapy, if indicated. Data on M-CHAT-R from children aged 18-30 months suggests that it is best used in a clinical setting and that it has low sensitivity (many false-negatives) but good specificity (few false-positives). However, the M-CHAT-R and other screening tools still present challenges in overall effectiveness and widespread adoption.

Few pediatricians (8 to 28%) conduct ASD-specific screening, citing a lack of familiarity with ASD screening and limited time during well-care visits as common deterrents. These barriers may arise because of inadequate training and experience in identifying more nuanced autistic features in young children, and the use of antiquated, inefficient diagnostic instruments and procedures. For example, in medical practices that use the M-CHAT-R screener, the only tool that is clinically validated, a paper questionnaire is typically given to parents in the waiting room. It is later scored in the office after the check-up by the pediatrician is over, and the parents and child have gone home. False positives and negatives occur because of rater bias or because of inadequate understanding of ASD and its symptoms, or because parents may not understand the questions or rush through the questionnaire, or because they may be inclined to just provide a random answer. If a follow-up interview is required or a referral for diagnosis indicated, someone in the doctor's office must contact the family to schedule the follow-up visits. Too often, the parents cannot be reached or do not return, especially if the family is of lower socioeconomic status, and no follow-up interview or referral takes place.

For example, researchers who examined the feasibility of ASD screening for all toddlers during a six-month period at a busy community pediatric practice in Utah used the M-CHAT-R and did follow-ups by telephone. The parents of 24 percent of the children could not be reached. Barriers to screening and surveillance of ASD at the earliest possible age arise because of inadequate training and experience in identifying more nuanced autistic features in young children, and the use of antiquated, inefficient diagnostic instruments and procedures. Consequently, the screening and surveillance process is haphazard and unreliable, and goes against AAP protocols for standardized screening and surveillance.

An additional public health issue is that the average age of ASD diagnosis, already too high, is even higher in minority and low-socioeconomic status children. Current screening tools show lower specificity and sensitivity with children from these demographics.

### SUMMARY OF THE INVENTION

New tools are disclosed herein to identify, detect, and diagnose neurodevelopmental disorders. In many of these disorders, early detection and early therapeutic intervention can make a substantial improvement in the treatment outcome.

In an embodiment, a database of video clips is provided, with a set of video clips depicting normal or abnormal behavioral patterns of an activity, and the abnormal behavior patterns are correlated to validated behaviors for the activity that characterize a neurodevelopmental disorder. Each video clip has metadata with indexing information describing the contents of the video clip. The normal and abnormal patterns for a given activity depicted in the video clips can be compared to a test subject performing the same activity to permit an observer to objectively assess the extent of a behavioral abnormality, if any, in the test subject.

Typically, the test subjects are children ranging in age from birth to about 21 years old, but the video clip library can also be constructed for adult onset neurodevelopmental disorders such as senile syndromes.

In an embodiment, a method of detecting or diagnosing a behavioral abnormality caused by a neurodevelopmental disorder in a test subject is provided, using a database of video clips that depict normal or abnormal behavioral patterns of a person engaged in an activity, wherein the abnormal behavior patterns are correlated to validated behaviors for the activity that characterize a neurodevelopmental disorder. Each video clip may have metadata with indexing information describing the contents of the video clip. An observer observing the behavior of the test subject performing an activity depicted in the video clip can objectively assesses the extent of a behavioral abnormality in the test subject.

This method may be used to detect the presence of a neurodevelopmental disorder, by allowing an observer to see examples of typical behaviors that present in various neurodevelopmental disorders, and to compare these behaviors to the behavior of the test subject. The test subject here is a person with an observer. The observer here may be the parent of a child who is the test subject, or a professional observer such as a physician, therapist, or other person trained to work with patients with neurodevelopmental disorders.

The method may also be of value in the differential diagnosis of neurodevelopmental disorders by professional observers.

In another embodiment, a natural history may be compiled, with a database of video clips depicting normal or abnormal behavioral patterns of a subject performing one or more activities, wherein the abnormal behavior patterns are correlated to validated behaviors for the activities that characterize a neurodevelopmental disorder. The video clip database includes series of identifiable individuals performing the activities, wherein each individual is depicted performing the activity two or more times at identified intervals during a timeframe from onset of disease to a cure or death of the identifiable individual.

In another embodiment, a method of assessing the progress of a neurodevelopment disorder using a natural history of a test subject having a neurodevelopmental disorder is provided, using a database of video clips depicting behavioral patterns of a subject having a neurodevelopmental disorder performing one or more activities typical for a neurodevelopmental disorder. The video clip database shows an individual performing the activity at a baseline time (or date) and at least one more time (or date) at an identified interval of the progression of the neurodevelopmental disorder, during a timeframe from onset of the disorder to a cure or death of the identifiable individual. A professional observer can use this data to assesses the extent of the behavioral abnormality in the test subject. The baseline date establishes an early detection of a behavior (either normal or abnormal), and the additional video clips at identified intervals show how the behavior changed over time, i.e., improved or deteriorated.

The critical advantage of assessing children at an early age is to identify the presence of a neurodevelopment disorder in order to initiate early treatment, which in many cases can be of enormous benefit in allowing patients to lead better (and in some cases, normal) lives, as measured by cognitive outcome, ability to attend school, learning, and social functioning. Improved functional outcomes help reduce the burden to families, school systems, and society.

### DESCRIPTION OF THE DRAWINGS

FIG. 1 shows a flow chart of an embodiment of this invention.

### DETAILED DESCRIPTION

The instant invention includes a database of video clips that are of value in the identification, detection, or diagnosis of persons who may have a neurodevelopmental disorder, such as a neurodevelopmental disorder described in the DSM-5. Some exemplary disorders are autism spectrum disorder (ASD), attention-deficit/hyperactivity disorder (ADHD), Fragile X Syndrome, Angelman Syndrome, Mucopolysaccharidosis type I & II, and Gaucher's disease. The inventive databases may be used to for comparisons and assessments, and to establish a natural history of the progression of a neurodevelopmental disorder in a particular individual having a neurodevelopmental disorder, which can be of value in designing treatments and other therapeutic interventions.

As used herein, the terms “database” and “library” are equivalent and may be used interchangeably.

In an embodiment, this invention provides a database of video clips, a wherein each video clip depicts normal or abnormal behavioral patterns of an activity, wherein the abnormal behavior patterns are correlated to validated behaviors for the activity that characterize a neurodevelopmental disorder. Each video clip has metadata providing indexing information describing the contents of the video clip. The normal and abnormal patterns for a given activity can be compared to a patient performing the same activity to permit an observer to assess the extent of a behavioral abnormality in the test subject. By the term “test subject,” is meant the person under evaluation with an observer, such as a physician, parent (of a child who is the test subject), or other professional trained in the identification or treatment of neurodevelopmental disorders.

The activities depicted in the video clips in the database may include ordinary activities, such as walking, talking, interacting with others, playing with toys, eating, and the like. The activities may also include typical activities that manifest in neurodevelopmental disorders, for example showing how autistic children avoid eye contact. In many embodiments, the subjects performing the activities are children. The children may be aged from birth to 21 years. Alternatively, the subjects performing the activities may be or adults.

The observer may be a parent or legal guardian (or other caregiver) of a child being assessed for a neurodevelopmental disorder. Alternatively, the observer may be a trained professional, such as a physician, a therapist, a nurse, or other person specifically trained to assess and treat neurodevelopmental disorders.

The video clips are typically fairly short, and generally range (i.e., the playing time) from about 15 seconds to about 20 minutes, or from about 30 seconds t0 about 2.5 minutes. But longer video clips are within the scope of the invention.

The neurodevelopmental disorder can be any neurodevelopmental disorder described in the DSM-5. In embodiments, the neurodevelopmental disorders include autism spectrum disorder (ASD), attention-deficit/hyperactivity disorder (ADHD), Fragile X Syndrome, Angelman Syndrome, Mucopolysaccharidosis type I & II, and Gaucher's disease. The neurodevelopmental disorder may also include adult onset disorders, such as senile disorders like Alzheimer's Disease.

The metadata associated with each video clip may be used to search for various activities depicted in the video library. For example, an observer may want to view eating behaviors. The metadata is thus used to quickly direct observers to relevant video clips.

The activities depicted in the video clips may be typical developmental milestones in children, for example, the behavior at children starting to speak at age one year, or remove clothing at age 18 months. For example, normal children are expected to speak simple words by age 12 months, which is a developmental milestone. When depictions of normal children at a given developmental milestone are compared to a patient, an observer can objectively assess neurodevelopmental abnormalities, if any. If present, early intervention and treatment can avert much of the impairment for certain neurodevelopmental disorders. Thus, there are significant advantages to early detection and diagnosis.

In another embodiment, a method is provided of diagnosing a behavioral abnormality caused by a neurodevelopmental disorder in a test subject, including a database of video clips, wherein each video clip depicts normal or abnormal behavioral patterns of an activity in humans. The abnormal behavior patterns are correlated to validated behaviors for the activity that characterize a neurodevelopmental disorder. Each video clip has metadata, which includes indexing information describing the contents of the video clip. An observer observes the behavior of the test subject performing the activity, and the normal and abnormal patterns for the activity presented in the video clips is compared to the test subject performing the same activity, and the observer objectively assesses the extent of a behavioral abnormality in the test subject.

By the phrase “objectively assess the extent of a behavioral abnormality,” it is meant that the observer can directly compare subject behaviors to validated behaviors, and confirm the presence of the behavior objectively. In some cases, the behaviors may be graded or scored to quantify the degree of impairment. In an embodiment, the video clips are validated by experts in the study of neurodevelopmental disorders. Thus experts can confirm, for example, that a video clip depicting a child with Angelman Syndrome flapping their arms is in fact a typical behavior associated with Angelman Syndrome.

In an embodiment, a natural history may be included in a database of this invention. Such a database may depict a series of identifiable subjects performing various activities, wherein each identifiable subject is depicted performing an activity two or more times at identified intervals. The intervals will be during a timeframe from onset of disease to a cure or death of the identifiable individual. This timeframe may range from months to many years. For example, the walking behavior of a person may be depicted on an annual basis over several years. This can show, for example, how treatment (or the absence of treatment) can affect the progression of the disorder in a particular person.

In another embodiment, a method of assessing the progress of a neurodevelopment disorder is provided, with a natural history, in a test subject having a neurodevelopmental disorder. A database of video clips is provided depicting behavioral patterns of a subject having a neurodevelopmental disorder performing one or more activities, wherein the behavior patterns are correlated to typical behaviors for the activities that characterize a neurodevelopmental disorder. The video clip database uses a series of identifiable individuals performing the activities, wherein each individual is depicted performing the activity at a baseline and at least one more time at an identified interval of the progression of the neurodevelopmental disorder, during a timeframe from onset of the neurodevelopmental disorder to a cure or death of the identifiable individual. Using this database, a professional observer observes the behavior of a test subject performing an activity in the database at times correlated to the identified intervals during the progression of the disease, and the normal and abnormal patterns for the activity presented in the video clips are compared to the test subject, and the observer objectively assesses the extent of the behavioral abnormality in the test subject.

In the natural history embodiments of this invention, the baseline is a video at a time or date that establishes an early detection of a behavior, which may a depiction of a normal or abnormal behavior. The additional video clips at identified intervals show how the behavior changed over time, i.e., improved or deteriorated. This can be valuable information to assess, for example, an improvement which may imply a successful therapeutic intervention. Alternatively, the change over time can illustrate a deterioration and permit caregivers to make a qualitative or quantitative assessment of the deterioration in the subject.

In various embodiments, the video clips may be about 15 seconds to about 20 minutes in length, or about 15 seconds to about 5 minutes in length, or about 15 seconds to less than five minutes. For example, many of the video clips are between about 45 seconds and about 150 seconds.

In an embodiment, the subjects depicted in the video clips are children aged from birth to about age 21 years. Video clips also include adult subjects, particularly as part of natural history libraries or databases intended for use with adult onset disorders.

A database of video clips has been constructed. The video clips in the video clips were produced from a much larger set of video data of normal and abnormal children in a wide variety of situations. Some of the video data was filmed in controlled clinical settings, but other video data was filmed in homes, schools, playgrounds, parks and other natural environments in which children spend their time. In fact, the video clips in the inventive database are culled from hundreds of hours of video of persons (mostly children) either as normal or with identified neurodevelopmental disorders.

The larger set of video data was reviewed by panels of experts comprising psychiatrists, psychologists, and experienced autism therapists to produce the individual video clips depicting clinically significant behaviors. Each video clip was also assigned metadata, also termed indexing information, describing the contents of the video clip.

For example, the instant invention provides a video-enhanced and automated version of the most widely used and clinically validated ASD-specific screener, the M-CHAT-R. The instant invention is termed the “Interactive Modified Checklist for Autism in Toddlers” (iM-CHAT). The iM-CHAT consists of a library of video clips of children ages birth to about five years of age, that may be presented on a website designed to be accessed by stakeholders concerned about autism—parents, pediatric healthcare providers, special education teachers, researchers, therapists and others—to see what these early warning signs look like and to learn about the therapies that can change an affected child's life. This website may provide an education and awareness platform for stakeholders to help inform and educate how to recognize the symptoms of ASD and use that information to facilitate engagement with their healthcare provider.

In an embodiment, the iM-CHAT is a method of diagnosing behavioral disorders in test subjects. The test subjects are children, aged about 16 months to about 30 months. The method includes the steps of a parent or care-giver being posed a series of questions about the behavior of the test subject in series of routine tasks, such as verbalization, pointing at objects, playing with toys, interacting with others, and eye contact with others. This list is not intended to be limiting.

The method further includes video clips illustrating normal and abnormal behavior for the tasks being assessed. In the method, the parent or caregiver can compare the behavior of the test subject and the normal and abnormal behavior illustrated in the video clips. The video clips may have metadata comprising indexing information. This comparison of a test subject to the illustrated and validated behaviors in the video clips allows the parent or caregiver to provide much more accurate answers to the iM-CHAT questions than in the conventional M-CHAT-R that relies entirely on written descriptions. Based on the comparisons of normal and abnormal behavior viewed in the video clips, and on the parent/care-givers observation and knowledge of the test subject, the parent/care-giver completes the questionnaire of approximately 20 questions.

The answers are used to compute a risk factor of the test-subject child for having a neurodevelopmental disorder. If the test-subject child is indicated as being at high risk, the child will be referred to a more detailed follow-up evaluation, and treatment if necessary.

In addition, other embodiments of this invention allow the iM-CHAT test to be performed in advance at home or in a waiting room so a practitioners time is not consumed with a task that can be easily performed by the parent or lower skilled medical assistant, unless the iM-CHAT results indicate a likelihood of a behavioral abnormality. Thus, in an important aspect of this invention, the identification of children at-risk for neurodevelopmental disorders is more efficient with the instant invention than with prior art methods.

Several behavioral abnormalities are amenable to detection by this invention. Generally, the behavioral abnormalities are neurodevelopmental disorders (as defined in the DSM-5), and may also be neurological conditions or metabolic disorders with neuro psychiatric symptoms. The condition may be Angelman syndrome, Mucopolyssacaridosis type I & II, Gaucher's disease, and autism spectrum disorder, attention-deficit/hyperactivity-disorder and many other disorders. Other neurodevelopmental disorders as described in the DSM-5 may also be amenable to use with the iM-CHAT video clip library and method.

Another example of the use of the inventive methods is termed the “SEE SYSTEM.” The SEE system (Symptom-surveillance, Education, Enhancement) is a collaboration between medical professionals, documentary filmmakers, software application designers, and web developers to create a platform to deploy video as a tool for doctors, patients, and their families. The SEE system involves intensive use of video to help capture the ambiguities and complexities of disorders with behavioral symptoms. In collaboration with expert practioners in neurodevelopmental disorders, patients with diverse backgrounds, ages, and levels of disease severity in various settings (clinic, home, school, and others) were extensively videotaped. This video was edited into multiple formats—from feature length documentaries to short observational clips, adding text descriptions, keywords, and other metadata. The videos are then integrated into the SEE digital environment to facilitate the creation of video-enhanced screening tools and assessment tests, educational features for families, and continuing medical education courses for health care providers.

In an embodiment, the library of video clips may be accessible to stakeholders such as parents, pediatric healthcare providers, special education teachers, researchers, therapists and others on a website that allows stakeholders to see what these early warning signs look like and to learn about the therapies that can change the life of a child's afflicted with a neurological disorder.

In an embodiment, the website may include an interactive tool that allows parents to track the milestones of their child—a personal health record feature that offers two-way communication between practitioner and patient/family. The website may also include tools to assist pediatricians in observing signs of autism during the 18 and 24 month well visits. The website may also include a special area for pediatricians only with Continuing Medical Education (CME) materials for practitioners on detection and therapy options for autism. The website may also include a searchable library of video clips for parents and professionals illustrating various symptoms and signs of autism or other neurological disorders.

For example, a symptom of autism is that a child does not point to something with one finger. In an embodiment, a video would show a normal child pointing to an object of interest with one finger. A companion video may show an autistic child engaging in some other action that is not pointing, when pointing or attention would be a more appropriate response. The video library may be indexed with such features so that parents and professionals can search for specific activities and view video clips of normal and abnormal behaviors for comparison with their child or patient. In an embodiment, the child or patient is referred to herein as a “test subject.”

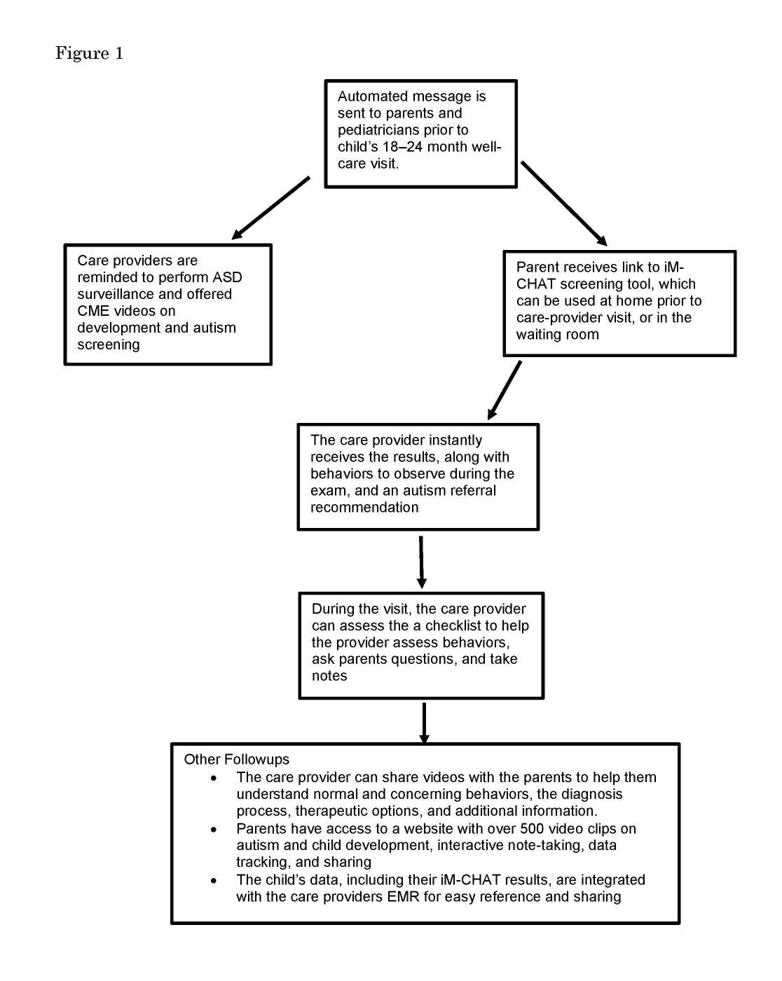

The instant invention of video libraries and diagnostic methods for pediatric healthcare providers that can be integrated into an electronic medical records (EMR) and/or information systems in a medical practice. The practitioner may receive a pre-well-visit reminder about an upcoming ASD screening (see FIG. 1). Parents may also receive an automatic reminder (by phone, text or email) and be directed to a website to learn about ASD. The website may have an implementation of iM-CHAT or the SEE System that parents can use online at home, or in the medical practice waiting room on a laptop or tablet, and submit the results will be sent to the doctor or other care-provider. The iM-CHAT informs and educates with short videos of children demonstrating the behaviors in question, provides an audio voice asking the questions, and is scored automatically. These features are expected to improve the overall accuracy of the parent report, and may be helpful to parents with low literacy or limited English comprehension.

The care providers that may use this invention include medical doctors, in particular (but not necessarily) pediatricians, trained medical professionals such as nurses or medical technicians, parents, special education teachers, researchers, therapists. Regardless of the nature of a care-provider's background, the care-provider will need some degree of training in identifying warning signs of autism or other neurological disorders for further follow-up.

The iM-CHAT consists of a list of 20 questions about the child's behavior to be answered by the parent. Many pediatricians continue to report a lack of familiarity with ASD screening tools as well as a lack of time to conduct screening as the primary barriers to screening and surveillance of autism. The iM-CHAT's video illustrations clarify the “missing milestones” of ASD in toddlers and the results are available immediately during the busy well visit, breaking through the primary barriers keeping doctors from screening for autism. Each doctor in the practice will have a customized dashboard with the results of each patient's assessment and the ability to track any changes over time from one well visit to the next.

In an embodiment, the iM-CHAT may be provided for pediatric health care providers that follows the protocols for screening and surveillance by healthcare professionals for autism in toddlers of the American Academy of Pediatrics. On this basis, the iM-CHAT or SEE System may be licensed to practitioners on a subscription basis, using the software as a service (SaaS) model (annual license fee plus monthly fee per practitioner). The software may be internally produced, centrally hosted on dedicated servers (web and database servers) by a contracted provider, accessed via web browsers and designed to integrate at the patient-level with EMRs, in private practices, hospitals for clinics. The software may include all the features on the website with the addition of a clinically validated screening tool, the iM-CHAT.

The video library component of iM-CHAT was developed by experts who filmed over 500 hours of video content of normal children and autistic and other impaired children performing routine activities, including activities that are assessed in the M-CHAT-R. This video content was edited to a library of approximately 550 video clips that vary in length from about 5 seconds to about 60 minutes in length, although most are less than about five minutes in length. In an embodiment, the video clips are designed to be viewed in pairs, of a normal child and an impaired (for example, an autistic) child performing the same behavior. By viewing the video clips, the parent or care-giver can more easily assess the specific behaviors associated with autism or other impairments. By using the iM-CHAT tool in this way, parents or other care-givers can more accurately determine if a child needs a follow up evaluation and therapy for a disorder such as autism or other impairment.

In an embodiment, each video clip is indexed with attributes about the activities depicted in the video clip, and whether the video clip depicts the behavior of normal or abnormal subjects. For example, a video clip may depict a child interacting with a toy rolled on wheels across a table. A normal child will take a great interest in such a toy and with the adult working with the child. A separate video clip may show an autistic child in the same setting with the same toy, but the autistic child may be indifferent to the toy, and try to climb out of his/her seat. In such an example, the indexing may include notations of a child interacting with a rolling toy, and whether the video depicts normal or abnormal behavior. Table 1 shows representative indexing terms that may be used in the inventive system.

TABLE 1
Examples of indexing terms associated with video clips.

| | |
|---|---|
| Emotion | flattened affect |
| General Behaviors | tantrums |
| | screaming |
| | lack of inhibition |
| Learning | classroom strategies |
| | preference for structure |
| | school readiness skills |
| | reading |
| Nonverbal Behaviors | eye contact |
| Used in Social | facial expressions |
| Interaction | |
| Play | interest in books |
| Social Interaction | spontaneous imitation |
| | lack of interest in other children |
| | interest in other children |
| Therapies | imitation |
| Verbal Communication | receptive language |
| Skills | expressive language |
| | lack of expressive language |

As illustrated in Table 1, in an embodiment, the indexing terms may have two parts, a broad term and a narrow term. For example, “Verbal Communication Skills” is a broad term, and within that broader term there may be optional narrower terms, for example “receptive language.” The use of these terms allows care givers to search for specific video examples of a given behavior or technique.

In an embodiment, the indexing terms may be compiled into a searchable database that can be searched on a computer system containing the video clips. For example, this could be a website, or it could be in a EMR system, or a stand-alone computer station. In such an embodiment, a user can search for a specific term, for example “eye contact,” and be presented with a list of video clips demonstrating eye contact, and possibly failure to establish eye contact.

The M-CHAT-R test is a clinically validated standard of care for assessing autism or other neurodevelopmental disorders in children. The M-CHAT-R consists of about 20 yes/no questions. In an embodiment, the iM-CHAT will provide video clips demonstrating normal and abnormal behavior for each of the questions in the M-CHAT-R test. In an embodiment, the metadata of each video clip is correlated with a clinically validated neurodevelopmental impairment assessment method.

The instant invention is also expected to improve healthcare and the diagnosis and assessment of neurodevelopmental disorders in disadvantaged minority and low-socioeconomic status children. The average age of ASD diagnosis, already often too high for optimal treatment, is even higher in these demographics. This is attributable to lower quality health-care in disadvantaged demographics, and the various stresses of poverty. By improving the efficiency of screening as described the instant invention, children at-risk for neurodevelopmental disorders can be flagged more efficiently and given treatment.

### Examples

### Example 1. Video Database

A library was created of 549 video segments and educational documentaries that show the subtle behavioral signs of ASD, the diagnostic process, and the value of early therapy, along with a suite of video-enhanced interactive tools for doctors to support surveillance and screening during well visits. The core component of the program, the iM-CHAT, is an audio-video enhanced, interactive, automatically scored version of the Modified Checklist for Autism in Toddlers (M-CHAT-R). The iM-CHAT features video examples of the behaviors and audio of the questions and is scored automatically. It also integrates the Follow-Up Interview to appear immediately afterwards if the answers are inconclusive.

These features and tools may be adapted and modified to be available at the right time and place during the pediatric care workflow, making it easy for practitioners to follow the protocols recommended by the American Academy of Pediatrics (AAP) for the detection of ASD in children under three, by eliciting parent concern, maintaining a developmental history, making informed observations of the child, administering an ASD-specific screener at the 18 and 24 month well visits, and referring a child suspected of possible ASD for a diagnosis and intervention as early as possible.

### Example 2. Clinical Validation

A clinical trial will be conducted to demonstrate that the iM-CHAT, with audio and video enhancements, performs as well or better, psychometrically, than the most recent version of the paper M-CHAT-R.

About 3,000 children will be enrolled in in a multi-center trial. The children will be assigned to an iM-CHAT screening (according to the instant invention), or to a conventional M-CHAT-R screening. The screening scores will be independently evaluated by at least one expert in the field and additional follow-up evaluations may be performed. The iM-CHAT will be compared to the M-CHAT-R to assess the extent of false positives, false negatives and other relevant factors.

For a similar study, see Diana L. Robins, Karis Casagrande, Marianne Barton, Chi-Ming A. Chen, Thyde, Dumont-Mathieu and Deborah Fein, “Validation of the Modified Checklist for Autism in Toddlers, Revised With Follow-up (M-CHAT-R/F),” Pediatrics 2014; 133; 37; originally published online Dec. 23, 2013. DOI: 10.1542/peds.2013-1813.

1. A database of video clips comprising
a. a set of video clips wherein each video clip depicts normal or abnormal behavioral patterns of an activity, wherein the abnormal behavior patterns are correlated to validated behaviors for the activity that characterize a neurodevelopmental disorder;
b. wherein each video clip has metadata associated therewith, wherein the metadata comprises indexing information describing the contents of the video clip; and
c. wherein the normal and abnormal patterns for a given activity depicted in the video clips can be compared to a test subject performing the same activity to permit an observer to objectively assess the extent of a behavioral abnormality in the test subject.

2. The database of claim 1, wherein the video clips are between about 15 seconds to about 20 minutes in length.

3. The database of claim 1, wherein the video clips are between about 30 seconds and about 2.5 minutes in length.

4. The behavioral abnormality of claim 1, wherein the abnormality is selected from a neurodevelopmental disorder described in the DSM-5 having unique atypical behavioral symptoms.

5. The behavioral abnormality of claim 1, wherein the abnormality is selected from autism spectrum disorder (ASD), attention-deficit/hyperactivity disorder (ADHD), Fragile X Syndrome, Angelman Syndrome, Mucopolysaccharidosis type I & II, and Gaucher's disease.

6. The library of claim 1, wherein the video clips are compiled into a database searchable by index terms.

7. The set of video clips of claim 1, wherein the activities are correlated to developmental milestones in age-matched individuals.

8. A method of detecting or diagnosing a behavioral abnormality caused by a neurodevelopmental disorder in a test subject, comprising
a. a database of video clips wherein each video clip depicts normal or abnormal behavioral patterns of an activity, wherein the abnormal behavior patterns are correlated to validated behaviors for the activity that characterize a neurodevelopmental disorder;
b. wherein each video clip has metadata associated therewith, wherein the metadata comprises indexing information describing the contents of the video clip;
c. wherein a professional observer observes the behavior of the test subject performing a given activity depicted in the video clips, and the normal and abnormal patterns for the activity are compared to the test subject performing the same activity, and the observer objectively assesses the extent of a behavioral abnormality in the test subject.

9. The method of claim 8, wherein the video clips are between about 15 seconds to about 20 minutes in length

10. The method of claim 8, wherein the video clips are between about 30 seconds and about 2.5 minutes in length.

11. The method of claim 8, wherein the behavioral abnormality is a neurodevelopmental disorder as defined in the DSM-5 having unique atypical behavioral symptoms.

12. The behavioral abnormality of claim 8, wherein the abnormality is selected from autism spectrum disorder (ASD), attention-deficit/hyperactivity disorder (ADHD), Fragile X Syndrome, Angelman Syndrome, Mucopolysaccharidosis type I & II, and Gaucher's disease.

13. The method of claim 8, wherein the assessment further comprises a standardized series of questions, and the answers to the questions are automatically scored.

14. The set of video clips of claim 8, wherein the activities in the video clips are correlated to developmental milestones in children less than 21 years, and the test subjects are children age-matched to the activities in the video clips.

15. The set of video clips of claim 8, wherein the activities in the video clips are correlated to developmental milestones in adults.

16. The method of claim 8, wherein the observer is a parent of the test subject, legal guardian of the test subject, or trained healthcare professional caring for the test subject.

17. A method of diagnosing a behavioral abnormality caused by a neurodevelopmental disorder in a test subject, comprising
a. a database of video clips comprising a set of video clips, wherein each video clip depicts normal or abnormal behavioral patterns of an activity in humans, wherein the abnormal behavior patterns are correlated to validated behaviors for the activity that characterize a neurodevelopmental disorder;
b. wherein each video clip has metadata associated therewith, wherein the metadata comprises indexing information describing the contents of the video clip;
c. wherein a professional observer observes the behavior of the test subject performing the activity, and the normal and abnormal patterns for the activity depicted in the video clips are compared to the age-matched test subject performing the same activity, and the observer objectively assesses the extent of a behavioral abnormality in the test subject; and
d. wherein the metadata assists the observer in establishing a diagnosis for the test subject.

18. A database of video clips comprising
a. a set of video clips wherein each video clip depicts normal or abnormal behavioral patterns of a subject performing one or more activities, wherein the abnormal behavior patterns are correlated to validated behaviors for the activities that characterize a neurodevelopmental disorder;
b. wherein the video clip database comprises video clips of a series of identifiable individuals performing the activities, wherein each individual is depicted performing the activity two or more times at identified intervals during a timeframe from onset of disease to a cure or death of the identifiable individual.

19. A database of video clips for a test subject comprising
a. a set of video clips wherein each video clip depicts normal or abnormal behavioral patterns for one or more activities, wherein the abnormal behavior patterns are correlated to validated behaviors for the activities that characterize a neurodevelopmental disorder;
b. wherein the behavior of the test subject performing the activities is compared to the same test subject performing the same activity at another time, and the observer objectively assesses the extent of a behavioral abnormality in the test subject.

20. The behavioral abnormality of claim 18, wherein the abnormality is selected from a neurodevelopmental disorder described in the DSM-5 having unique atypical behavioral symptoms.

21. The behavioral abnormality of claim 18, wherein the abnormality is selected from autism spectrum disorder (ASD), attention-deficit/hyperactivity disorder (ADHD), Fragile X Syndrome, Angelman Syndrome, Mucopolysaccharidosis type I & II, and Gaucher's disease.

22. The library of claim 18, wherein the video clips are compiled into a database searchable by index terms.

23. A method of assessing the progress of a neurodevelopment disorder in a test subject having a neurodevelopmental disorder, comprising
a. a database of video clips wherein each video clip depicts behavioral patterns of a subject having a neurodevelopmental disorder performing one or more activities, wherein the behavior patterns are correlated to typical behaviors for the activities that characterize a neurodevelopmental disorder;
b. wherein the video clip database comprises a series of identifiable individuals performing the activities, wherein each individual is depicted performing the activity at a baseline and at least one more time at an identified interval of the progression of the neurodevelopmental disorder, during a timeframe from onset of the neurodevelopmental disorder to a cure or death of the identifiable individual; and
c. wherein a professional observer observes the behavior of the test subject performing an activity in the database at times correlated to the identified intervals during the progression of the disease, and the normal and abnormal patterns for the activity presented in the video clips are compared to the test subject, and the observer objectively assesses the extent of the behavioral abnormality in the test subject.

24. The method of claim 23, wherein the video clips are between about 15 seconds to about 20 minutes in length

25. The method of claim 23, wherein the video clips are between about 30 seconds and about 2.5 minutes in length.

26. The method of claim 23, wherein the behavioral abnormality is a neurodevelopmental disorder as defined in the DSM-5 having unique atypical behavioral symptoms.

27. The behavioral abnormality of claim 23, wherein the abnormality is selected from autism spectrum disorder (ASD), attention-deficit/hyperactivity disorder (ADHD), Fragile X Syndrome, Angelman Syndrome, Mucopolysaccharidosis type I & II, and Gaucher's disease.

28. The method of claim 23, wherein the assessment further comprises a standardized series of questions, and the answers to the questions are automatically scored.

29. The set of video clips of claim 23, wherein the activities in the video clips are correlated to developmental milestones in children less than 21 years, and the test subjects are children age-matched to the activities in the video clips.

30. The set of video clips of claim 23, wherein the activities in the video clips are correlated to developmental milestones in adults.
